(19) United States
(12) Patent Application Publication
(10) Pub. No.: US 2025/0375405 A1
(43) Pub. Date: Dec. 11, 2025

(54) N-LACTOYL-PHENYLALANINE FOR PREVENTING AND/OR TREATING ISCHEMIC HEART DISEASE AND METHOD FOR PREVENTING AND/OR TREATING ISCHEMIC HEART DISEASE

(71) Applicant: Harbin Medical University, Harbin (CN)

(72) Inventors: Yang ZHANG, Harbin (CN); Lei JIAO, Harbin (CN); Zheng LI, Harbin (CN); Zhiyuan DU, Harbin (CN); Manyu GONG, Harbin (CN); Yu WANG, Harbin (CN); Chenchen YIN, Harbin (CN); Tiantian GONG, Harbin (CN)

(73) Assignee: Harbin Medical University, Harbin (CN)

(21) Appl. No.: 18/940,875

(22) Filed: Nov. 8, 2024

(30) Foreign Application Priority Data
Jun. 7, 2024 (CN) 202410742403 .7

(51) Int. Cl. A61K 31/197; A61P 9/10

(52) U.S. Cl. CPC A61K 31/197; A61P 9/10

(57) ABSTRACT

A use of N-lactoyl-phenylalanine (Lac-Phe) in a preparation of a medicament for preventing and/or treating ischemic heart disease is provided. It is the first to demonstrate that Lac-Phe can improve the cardiac function in a mouse model of left anterior descending coronary artery stenosis, promote cardiac angiogenesis, improve the viability of hypoxic cardiomyocytes, and inhibit the expression of inflammatory cytokines in the heart, thereby playing an anti-myocardial ischemic injury role. Therefore, the Lac-Phe can be used to prepare a medicament for treating ischemic heart disease. A novel technical means for the treatment of ischemic heart disease is provided.

### CROSS REFERENCE TO THE RELATED APPLICATIONS

This application is based upon and claims priority to Chinese Patent Application No. 202410742403.7, filed on Jun. 7, 2024, the entire contents of which are incorporated herein by reference.

### TECHNICAL FIELD

The present disclosure relates to a novel use of N-lactoyl-phenylalanine (Lac-Phe), and in particular to a use of Lac-Phe in the prevention and/or treatment of ischemic heart disease. The present disclosure belongs to the technical field of pharmaceuticals.

### BACKGROUND

Currently, with the increase of the aging population in China, the incidence and mortality of cardiovascular diseases continue to increase. Ischemic heart disease has become one of three major cardiovascular diseases to cause death. In ischemic heart disease, persistent myocardial ischemia may lead to myocardial hypertrophy, myocardial infarction, and malignant arrhythmia and even lead to heart failure and is seriously life-threatening. At present, there are respective treatments for ischemic heart disease in the clinical setting, including drug therapy, percutaneous coronary intervention (PCI), and the like. However, these treatments still have disadvantages such as significant drug side effects and reperfusion injury risks. Therefore, how to provide a high-quality treatment strategy to a patient with ischemic heart disease, improve the prognosis of the patient, prevent the secondary myocardial ischemia or myocardial reperfusion injury, and reduce adverse drug reactions are clinical problems to be solved urgently for ischemic heart disease.

Studies have already shown that Lac-Phe is an exercise-inducible metabolite, and carnosine dipeptidase II (CNDP2) as a non-specific dipeptidase catalyses the formation of Lac-Phe in vivo. The supplementation of Lac-Phe can effectively reduce the food intake of experimental mice to suppress obesity. However, it is not clear whether the exercise-inducible endogenous molecule Lac-Phe can prevent and/or treat ischemic heart disease.

### SUMMARY

A technical problem to be solved by the present disclosure is to provide a medicament that can prevent and/or treat ischemic heart disease.

In order to achieve the above objective, the present disclosure adopts the following technical means.

The present inventors of the present disclosure have found through research that the endogenous molecule Lac-Phe can promote angiogenesis, inhibit an inflammatory response, and improve the viability of hypoxic cardiomyocytes, thereby alleviating a cardiac injury caused by ischemia. Therefore, the Lac-Phe has promising application prospects in the prevention and/or treatment of ischemic heart disease, including coronary heart disease, myocardial infarction, myocardial ischemia-reperfusion injury, myocardial hypertrophy, arrhythmia, and heart failure. In particular, the Lac-Phe can alleviate the coronary heart disease, myocardial infarction, myocardial hypertrophy, arrhythmia, and heart failure of an obese patient to improve a life quality of the obese patient.

On the basis of the above research, the present disclosure provides a use of Lac-Phe in a preparation of a medicament for preventing and/or treating an ischemic heart disease, where a structure of the Lac-Phe is as follows:

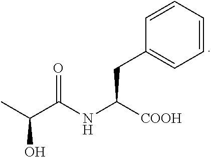 [SMILES: C[C@H](O)C(=O)N[C@@H](Cc1ccccc1)C(=O)O]

Preferably, the ischemic heart disease is selected from the group consisting of coronary heart disease, myocardial infarction, myocardial ischemia-reperfusion injury, myocardial hypertrophy, arrhythmia, and heart failure caused by myocardial ischemia.

Preferably, the ischemic heart disease is an ischemic heart disease in an obese patient.

Preferably, the Lac-Phe or a pharmaceutically acceptable salt thereof has an effect of alleviating cardiac dysfunction, heart inflammation, and cardiomyocyte death caused by myocardial ischemia.

Preferably, the medicament further includes a pharmaceutically acceptable carrier or excipient.

Preferably, the medicament is an oral preparation, a sublingual preparation, or an injection preparation.

Preferably, the medicament is one selected from the group consisting of a tablet, a capsule, a granule, an oral solution, and an injection.

Compared with the prior art, the present disclosure has the following advantages.

The present disclosure is the first to provide a novel use of the endogenous molecule Lac-Phe in the prevention and/or treatment of ischemic heart disease. The present disclosure has shown that the Lac-Phe can promote angiogenesis, inhibit inflammatory response, and protect the viability of cardiomyocytes suffering from ischemia, thereby alleviating an injury of ischemic heart disease. Therefore, this endogenous molecule has an effect of promoting the repair of the injury of ischemic heart disease, and can be used to prepare a medicament for preventing and/or treating ischemic heart disease. The present disclosure provides a medicament that can effectively prevent and/or treat an ischemic cardiac injury. The medicament can exhibit a therapeutic efficacy to recover the function of an ischemic heart by promoting angiogenesis, exerting anti-inflammatory effect, and restoring the viability of cardiomyocytes suffering from ischemic injury. The present disclosure provides a novel technical means for the prevention and/or treatment of ischemic heart disease.

### BRIEF DESCRIPTION OF THE DRAWINGS

FIG. 1A-FIG. 1C show that Lac-Phe improves the cardiac function in a mouse model of myocardial ischemia.

FIG. 2 shows that Lac-Phe improves the viability of hypoxic endothelial cells.

FIG. 3A-FIG. 3B show that Lac-Phe inhibits the apoptosis of hypoxic endothelial cells.

FIG. 4A-FIG. 4B show that Lac-Phe promotes the migration of endothelial cells.

FIG. 5A-FIG. 5C show that Lac-Phe promotes the tube formation of endothelial cells.

FIG. 6 shows that Lac-Phe improves the viability of hypoxic cardiomyocytes.

FIG. 7A-FIG. 7B show that Lac-Phe inhibits the release of inflammatory cytokines.

### DETAILED DESCRIPTION OF THE EMBODIMENTS

The present disclosure is further described below with reference to examples. It should be noted that the following examples are provided to merely illustrate the present disclosure rather than limit the present disclosure. All changes made by those skilled in the art according to the teachings of the present disclosure shall fall within the protection scope defined by the claims of the present disclosure.

### Example 1: Lac-Phe has a Protective Effect on the Cardiac Function in a Mouse Model of Myocardial Ischemia

The surgery for left anterior descending coronary artery ligation was performed on mice to establish a mouse model of myocardial ischemia (MI group). Mice in a sham surgery group (Sham group) were treated in the same procedures as those for the MI group, except that they did not undergo the surgery for ligation. Mice in the MI group were intraperitoneally injected with Lac-Phe (20 mg/kg), and then anesthetized 1 week later. An M-Mode echocardiogram was obtained under the guidance of B-Mode on a Doppler ultrasound Vevo2100 imaging system for small animals, and 10 or more cardiac cycles were recorded. Instrumental analysis software was used to measure various cardiac indicators of mice to assess the cardiac functions of the mice, including a left ventricular internal dimension systole (LVID), a left ventricular end systolic volume (LVESV), an inter-ventricular septum end-stolic thickness (IVS), an E/A ratio, an ejection fraction (EF), and a fraction shortening (FS).

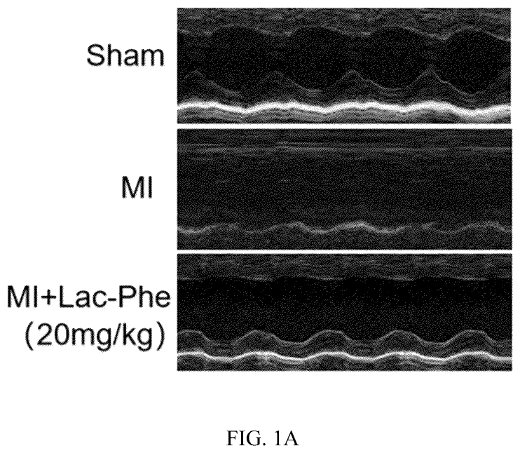

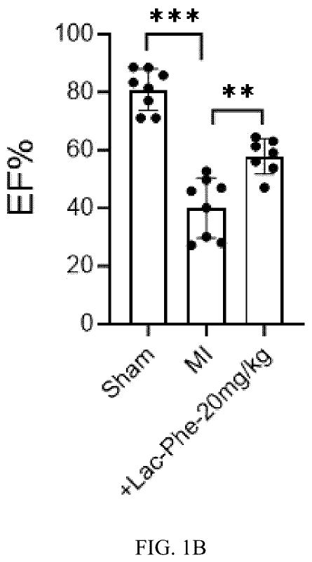

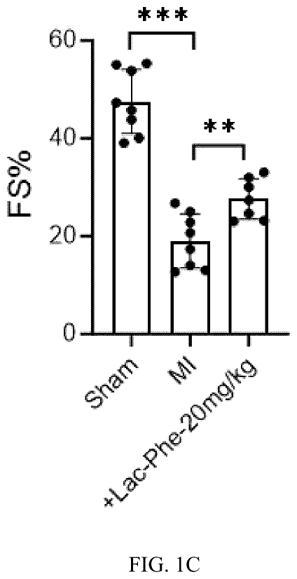

Experimental results are shown in FIG. 1A-FIG. 1C. It can be seen from the experimental results that the Lac-Phe has a significant improvement effect on the cardiac function in the mouse model of myocardial ischemia, compared with the MI group.

### Example 2: Effect of Lac-Phe on the Viability of Hypoxic Endothelial Cells

The Lac-Phe was added to human umbilical vein endothelial cells (HUVECs), and 8 hours later, the HUVECs were placed in a hypoxia incubator and cultured under hypoxia for 24 hours (N₂ 95%, CO₂ 5%). The cell viability was evaluated by Cell Counting Kit-8 (CCK8) method for the normal control group (CTRL), hypoxia group (Hypoxia), and Lac-Phe-treated hypoxia group.

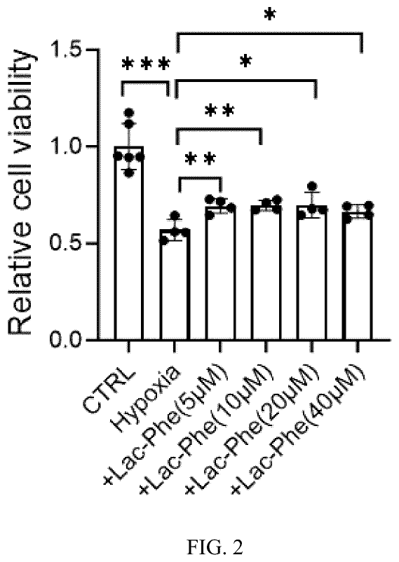

Experimental results are shown in FIG. 2. It can be seen from the experimental results that, after hypoxia, the cell viability of HUVECs is significantly reduced, and after Lac-Phe is added (final concentrations: 5 μM, 10 μM, 20 μM, and 40 μM), the cell viability of the hypoxic HUVECs is significantly improved.

### Example 3: Lac-Phe Inhibits the Apoptosis of Hypoxic Endothelial Cells

The Lac-Phe was added to HUVECs, and 8 hours later, the HUVECs were placed in a hypoxia incubator and cultured under hypoxia for 24 hours (N₂ 95%, CO₂ 5%). The cell apoptosis was determined by flow cytometry for the normal control group (CTRL), hypoxia group (Hypoxia), and Lac-Phe-treated hypoxia group.

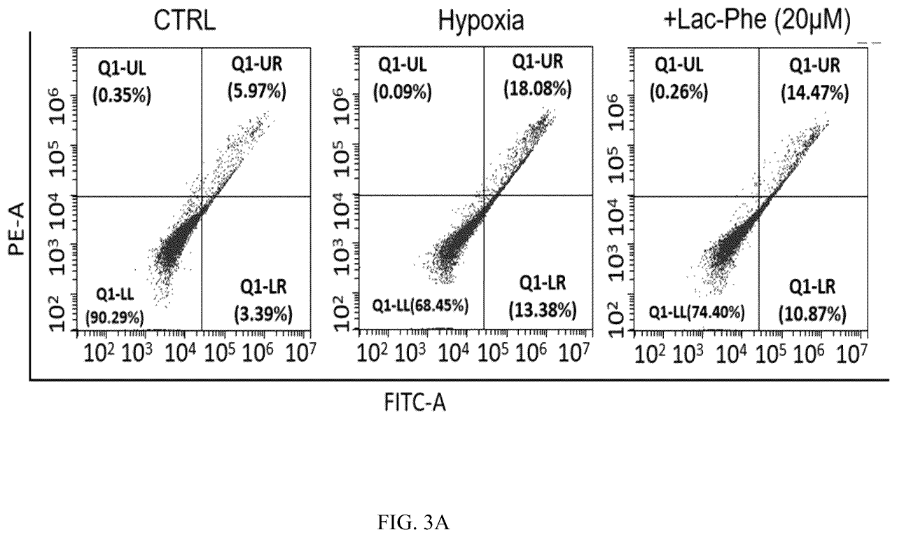

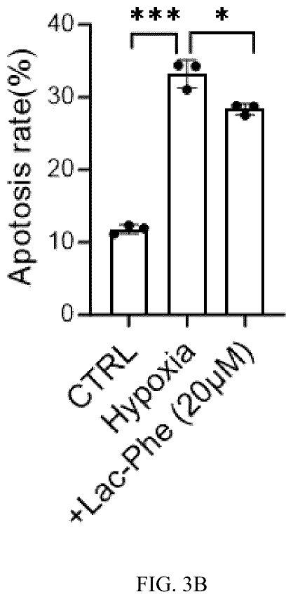

Experimental results are shown in FIG. 3A-FIG. 3B. It can be seen from the experimental results that, after hypoxia, the number of apoptotic HUVECs is significantly increased, and after Lac-Phe is added (final concentration: 20 μM), the number of apoptotic HUVECs is significantly reduced.

### Example 4: Lac-Phe has the Effect of Promoting the Migration of Endothelial Cells

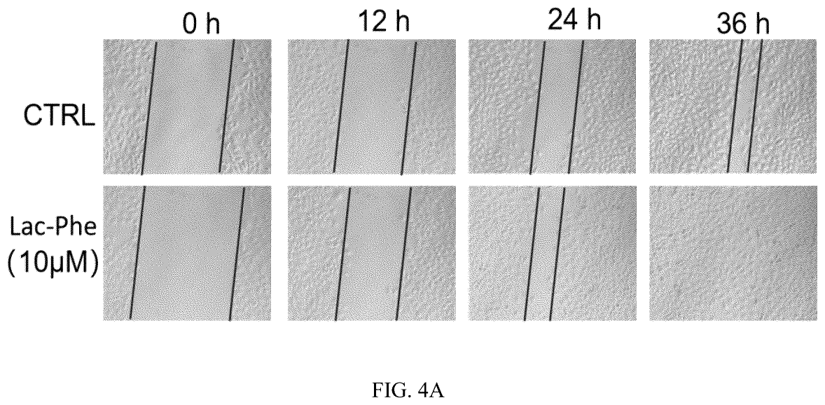

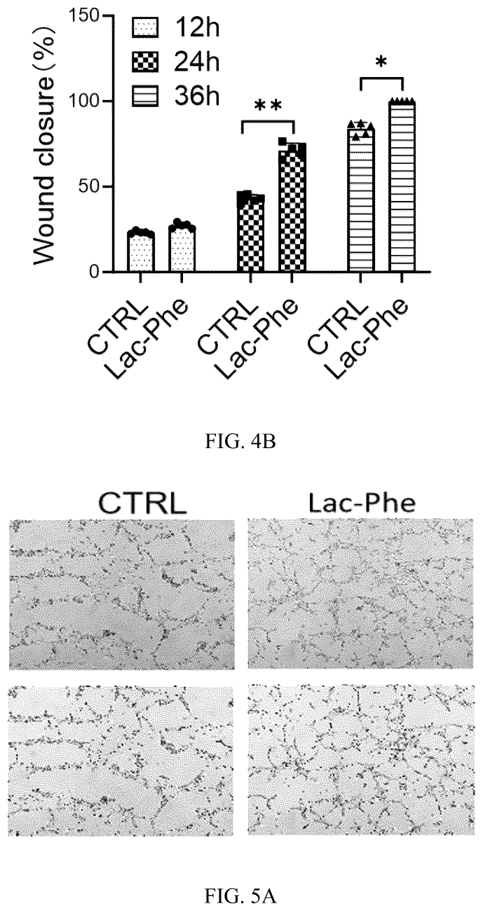

HUVECs were divided into 2 groups: a control group (CTRL, including 0.1% of dimethylsulfoxide (DMSO)), and a Lac-Phe-treated group (final concentration: 10 μM). Cell monolayers were wounded in wells of a cell culture plate along the central axis of each well by an autoclaved white pipette tip, suspended cells in each well were washed with phosphate buffered saline (PBS), and a medium without fetal bovine serum was added to each well. The wounds were photographed with an inverted microscope, that is, the wound area was recorded at 0 hour. The cells were then further incubated in a cell culture incubator at 37° C. for 12 hours, 24 hours, and 36 hours. Then, the wounds were photographed under the inverted microscope, that is, the wound area was recorded. The ImageJ software was used to quantitatively assess the wound closure (FIG. 4A-FIG. 4B).

Experimental results are shown in FIG. 4A-FIG. 4B. It can be seen from the experimental results that Lac-Phe has the effect of promoting the migration of endothelial cells.

### Example 5: Lac-Phe Promotes the Tube Formation of Endothelial Cells

HUVECs were divided into 2 groups: a control group (CTRL, including 0.1% of DMSO), and a Lac-Phe-treated group (final concentration: 20 μM). 24 hours after administration, the endothelial cell tube formation assay was performed.

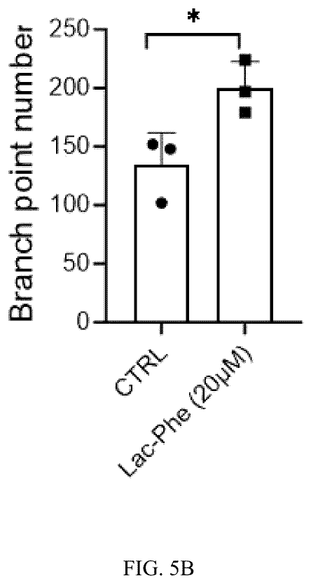

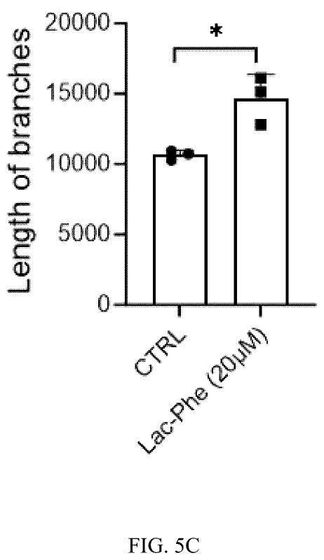

Experimental results are shown in FIG. 5A-FIG. 5C. It can be seen from the experimental results that Lac-Phe can increase the length of branches and the number of branch points formed from HUVECs (FIG. 5A-FIG. 5C). The above results also indicate that Lac-Phe has the potential to promote angiogenesis.

### Example 6: Lac-Phe Improves the Viability of Hypoxic Cardiomyocytes

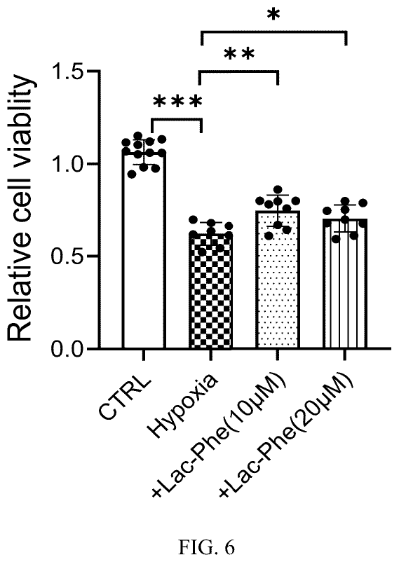

Primary mouse cardiomyocytes were divided into the following 4 groups: a control group (CTRL, including 0.1% of DMSO), a hypoxia group (a hypoxia treatment was performed for 24 hours), and hypoxia+Lac-Phe groups (cardiomyocytes were treated with Lac-Phe at final concentrations of 10 μM and 20 μM, respectively, and a hypoxia treatment was performed for 24 hours). The viability of cardiomyocytes was assayed by the CCK8 method. According to experimental results, the viability of cardiomyocytes is significantly reduced after the hypoxia treatment, and the administration of Lac-Phe can significantly alleviate the hypoxic injury of cardiomyocytes (FIG. 6).

Experimental results are shown in FIG. 6. It can be seen from the experimental results that Lac-Phe has the function of protecting cardiomyocytes against hypoxic injury.

### Example 7: Lac-Phe has the Anti-Inflammatory Effect

Macrophages (RAW264.7) were divided into the following 3 groups: a control group (CTRL, including 0.1% of DMSO), a lipopolysaccharide (LPS)-treated group (LPS-induced inflammatory responses in macrophages, a concentration: 100 ng/ml), and an LPS+Lac-Phe-treated group (Lac-Phe was added to LPS-induced macrophages at a final concentration of 20 μM). 24 hours after administration, RNA was extracted from cells in each group, and expression levels of pro-inflammatory cytokines IL-6 and TNFα in cells in each group were determined by quantitative real-time polymerase chain reaction (qRT-PCR).

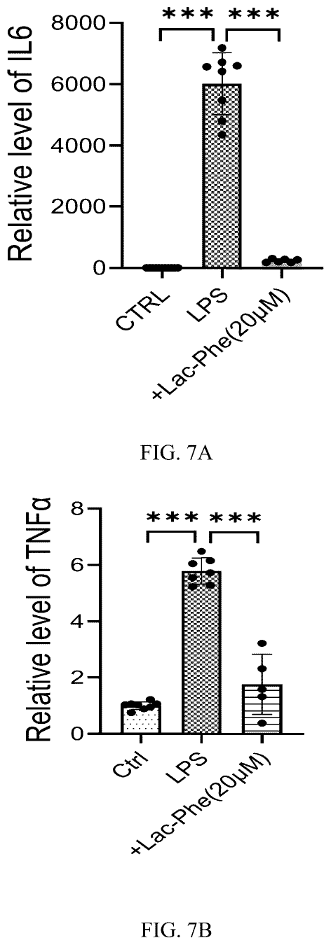

Experimental results are shown in FIG. 7A-FIG. 7B. It can be seen from the experimental results that expression levels of the pro-inflammatory cytokines IL-6 and TNFα are significantly increased in the LPS-induced group, and expression levels of the pro-inflammatory cytokines in the Lac-Phe-treated group are significantly decreased (FIG. 7A-FIG. 7B). The above results also indicate that Lac-Phe has the anti-inflammatory potential.

What is claimed is:

1. A method for preventing and/or treating an ischemic heart disease, comprising: administering to a subject a medicament comprising N-lactoyl-phenylalanine (Lac-Phe) or a pharmaceutically acceptable salt thereof in an effective amount,
wherein a structure of the Lac-Phe is as follows:

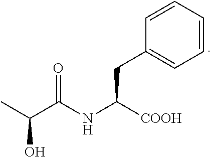 [SMILES: C[C@H](O)C(=O)N[C@@H](Cc1ccccc1)C(=O)O]

2. The method according to claim 1, wherein the ischemic heart disease is selected from the group consisting of coronary heart disease, myocardial infarction, myocardial ischemia-reperfusion injury, myocardial hypertrophy, arrhythmia, and heart failure caused by myocardial ischemia.

3. The method according to claim 2, wherein the ischemic heart disease is an ischemic heart disease in an obese patient.

4. The method according to claim 1, wherein the Lac-Phe or the pharmaceutically acceptable salt thereof has an effect of alleviating cardiac dysfunction, heart inflammation, and cardiomyocyte death caused by myocardial ischemia.

5. The method according to claim 1, wherein the medicament further comprises a pharmaceutically acceptable carrier or excipient.

6. The method according to claim 1, wherein the medicament is an oral preparation, a sublingual preparation, or an injection preparation.

7. The method according to claim 2, wherein the medicament is an oral preparation, a sublingual preparation, or an injection preparation.

8. The method according to claim 3, wherein the medicament is an oral preparation, a sublingual preparation, or an injection preparation.

9. The method according to claim 4, wherein the medicament is an oral preparation, a sublingual preparation, or an injection preparation.

10. The method according to claim 5, wherein the medicament is an oral preparation, a sublingual preparation, or an injection preparation.

11. The method according to claim 6, wherein the oral preparation, the sublingual preparation, or the injection preparation is one selected from the group consisting of a tablet, a capsule, a granule, an oral solution, and an injection.

12. The method according to claim 7, wherein the oral preparation, the sublingual preparation, or the injection preparation is one selected from the group consisting of a tablet, a capsule, a granule, an oral solution, and an injection.

13. The method according to claim 8, wherein the oral preparation, the sublingual preparation, or the injection preparation is one selected from the group consisting of a tablet, a capsule, a granule, an oral solution, and an injection.

14. The method according to claim 9, wherein the oral preparation, the sublingual preparation, or the injection preparation is one selected from the group consisting of a tablet, a capsule, a granule, an oral solution, and an injection.

15. The method according to claim 10, wherein the oral preparation, the sublingual preparation, or the injection preparation is one selected from the group consisting of a tablet, a capsule, a granule, an oral solution, and an injection.
